(19) United States
(12) Patent Application Publication
(10) Pub. No.: US 2008/0221612 A1
(43) Pub. Date: Sep. 11, 2008

(54) Internal tourniquet system

(76) Inventor: Keith J. Rose, Corpus Christi, TX (US)

Correspondence Address: Peter A. Shaddock II;Bowman Green Hampton & Kelly, PLLC, Suite 201, 501 Independence Parkway, Chesapeake, VA, 23320, US

(21) Appl. No.: 11/599,980

(22) Filed: Nov. 15, 2006

(51) Int. Cl. A61B 17/00

(52) U.S. Cl. 606/203

(57) ABSTRACT

An internal tourniquet system may comprise as many as eight tourniquets strategically positioned, two per extremity, so as to optimize the inversely proportional relationship between the force applied to the tourniquet and the blood flow from the laceration on the extremity farther from the body than the positioned tourniquet. Each garment is equipped with internal channels to position the tourniquets and each garment is equipped with removable covering flaps for ease of access to the tightening and securing mechanism on the tourniquets. Each garment may also be supplied with a lever or bar and a storage location on the garment in order to apply more torque if necessary to the tourniquet banded site. And each garment may also be supplied with such flaps that are able to secure the lever or bar in position, once to blood flow has ceased to an acceptable level.

### CROSS-REFERENCE TO RELATED APPLICATIONS

N/A

### STATEMENTS REGARDING FEDERALLY SPONSORED RESEARCH OR DEVELOPMENT

N/A

### REFERENCE TO A MICROFICHE APPENDIX

N/A

### BACKGROUND OF THE INVENTION

1. Field of the Invention

This invention generally relates to wearing apparel and particularly to garments and garments worn by users engaged in hazardous occupations or activities and where injury to the extremities is a possibility.

2. Description of the Related Art

The current tourniquet system is carried by a user or a medic for a group of users, or other groups in hazardous conditions; however, in this configuration, the entire group has to stay within each other's sightline and the hazardous area must remain small. In addition, the medic himself cannot be injured or the first aid kit lost or destroyed.

In addition, U.S. Pat. No. 2,702,551 issued to G. G. Hobson on Feb. 22, 1955 discloses garments with one tourniquet per extremity equipped to the outside of the garment. This configuration allows for tourniquet use, however, there is the risk of snagging to the user's garment, the risk that the injured user won't possess the force necessary to halt the blood loss when the tourniquet is placed in a less than optimal area, and only one choice in positioning the tourniquet thus having to cut off essential blood supply to more of the extremity than the injury requires. The configuration also doesn't allow for a method of securing the tourniquet once the required force is applied. While the patent additionally discloses a suggested alternate configuration wherein the tourniquet bands are placed inside the clothing, the patent doesn't disclose the method for securing the tourniquet bands, and plainly states that the large loops of the tourniquet should still be accessible through holes in the garments, thus destroying any attempt to maintain the smooth exterior of the user's garment.

In view of the above described deficiencies associated with tourniquet systems applied to garments, the present invention has been developed to alleviate these drawbacks and provide further benefits to a user. These enhancements and benefits are described in greater detail herein below with respect to several embodiments of the present invention.

### BRIEF SUMMARY OF THE INVENTION

The present invention in its several disclosed embodiments alleviates the drawbacks described above with respect to the current methods and systems for dealing with severe lacerations and incorporates several additional beneficial features. The general objective of the present invention is to provide a new and improved internal tourniquet system for garments worn by users in high risk activities. The garment may be a article of clothing or may be a specialized uniform or suit like a pilots pressurized suit or a wet suit. It might also be a protective covering worn over conventional clothing.

Currently, around 70% of all fatalities on the battlefield are users with extremity trauma who died of exsanguinations. Unfortunately, medical care is not always immediately available and the user or his buddy has to tend to his own or his friend's wounds. This has been the case over the last couple of years where use of improvised explosive devices has sharply increased. The users have armor that protects their torsos; however, since the extremities are left unprotected the users are more likely to suffer a severe laceration in those extremities. The large loss of blood from these lacerations can be avoided with the quick application of a well-placed tourniquet.

In some situations, as described above, the injured, weakened user may have to apply the tourniquet himself and the invention places the tourniquets in particular locations such that the least amount of pressure is required to arrest the largest amount of blood flow. On the upper extremities, the first tourniquet band's centerline must lie proximal to the superior position of the bicep and the second tourniquet band's centerline, on the same upper extremity, must be within about two centimeters below the elbow of the wearer. On the lower extremities, the first tourniquet band's centerline must lie within about two centimeters distal to the inguinal crease and the second tourniquet band's centerline must lie two centimeters distal to the tibial tuberosity.

The invention would add a much needed medical supply to each user's garment without sacrificing the comfort of the user, the weight of the garment, or the user's ability to move through his environment stealthily. The tourniquet is positioned inside the garment such that it will be in the optimal position for use and the webbing used to maintain the position of the tourniquet is light, and non-chafing. The tourniquet itself is made with tactical-grade nylon or a similar material and is positioned between the webbing and the interior of the garment. The tightening and locking mechanism is made of a light-weight metal or durable polymer or similar material and placed below the flap of the garment for easy access. The flap also acts to protect the tourniquet during times of disuse and acts maintain a smooth appearance to the user's garment.

The current invention could also be extremely useful in wetsuits, for implementation after shark attacks, or ski diving garments wherein the wearer is being dropped into an area not easily accessible by ground, or hazmat suits where the user might be in contaminated conditions. Variants could also include a pneumatic tourniquet that the wearer could trigger to apply increasing compressing pressure at a desired tourniquet location without any manual force or an attached transmitter that would activate when the internal tourniquet system is used to alert the medical field to the user's location and particular injury.

An object of this invention is to provide a garment having a total of eight tourniquets incorporated into the inner surface of the garment with webbing that is non-chafing and light; an object of this invention is to have tourniquets incorporated into the inner surface of the garment at strategic locations requiring the least amount of pressure applied to result in the lowest amount of blood loss from the extremity; an object of this invention is to provide a garment having tourniquets incorporated into the inner surface of the garment with flaps available at the site of each tourniquet's tightening and securing mechanism in order to allow for easy access to the tourniquet, to protect the tightening and securing mechanism from the environment during non-use and to maintain the smooth appearance of the user's garment. An object of this invention is to provide a garment with an additional lever or bar, while continuing to maintain the smooth appearance of the user's garment, to allow additional torque to be applied by the injured user or second party if necessary

The present invention will be more clearly understood from the following description of illustrative embodiments thereof, to be read by way of example and not of limitation in conjunction with the apparatus and the method described. The beneficial effects described above apply generally to the examples disclosed herein of the internal tourniquet system. The specific components and configurations through which these benefits are delivered will be described in detail herein below.

### BRIEF DESCRIPTION OF THE SEVERAL VIEWS OF THE DRAWINGS

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a basis for the claims and as a representative basis for teaching one skilled in the art to variously employ the present invention.

FIG. 1 is a perspective view disclosing the preferred embodiment of the internal tourniquet system formed in accordance with the present invention.

FIG. 2 is a perspective view disclosing the internal tourniquet system-equipped short-sleeved shirt formed in accordance with the present invention.

FIG. 3 is a vertical sectional view disclosing a suggested location for the storage location of the lever or bar.

FIG. 4 is a cross section of one particular embodiment of the internal tourniquet system formed in accordance with the present invention.

FIG. 5 is a cross section of one particular embodiment of the internal tourniquet system's channel formed in accordance with the present invention.

FIG. 6 is a cross section of one particular embodiment of the internal tourniquet system's channel formed in accordance with the present invention.

FIG. 7 is a perspective view of one embodiment of the tourniquet tightening and securing mechanism in its secured position.

FIG. 8 is a perspective view of the preferred embodiment of the tourniquet tightening and securing mechanism in its unsecured position.

FIG. 9 is a perspective view of one embodiment of the tourniquet tightening and securing mechanism in its unsecured position.

FIG. 10 is a perspective view of another embodiment of the tourniquet tightening and securing mechanism in its unsecured position.

### DETAILED DESCRIPTION OF THE INVENTION

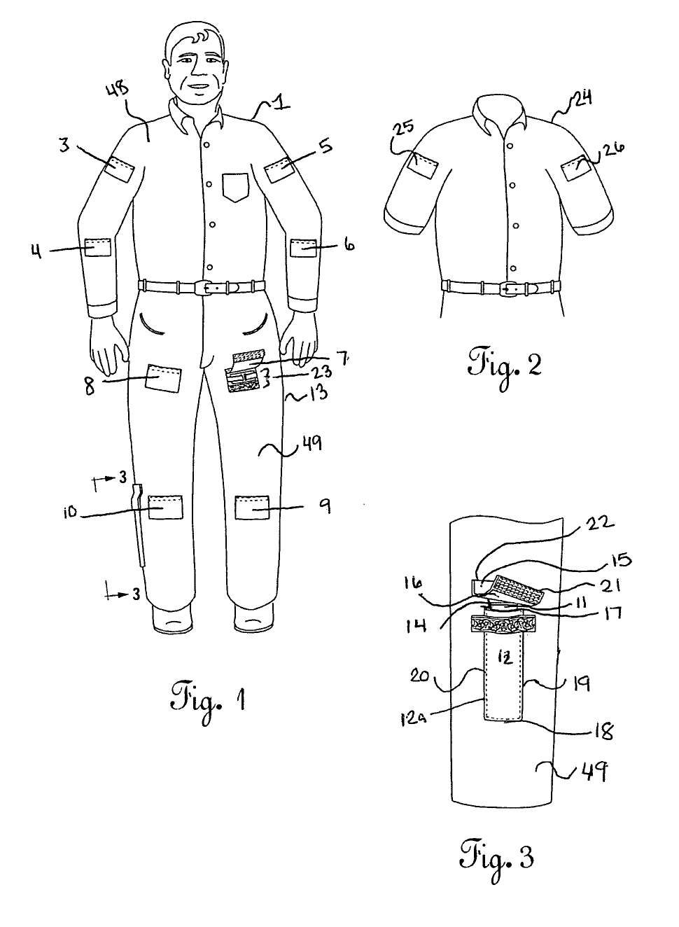

FIGS. 1 through 10 illustrate particular embodiments of the internal tourniquet system that is the current invention. FIG. 1 depicts the preferred embodiment of the current invention in a garment that comprises a full-sleeved, button-down shirt 1 with exterior surface 48 and two-pocket pants 13 with exterior surface 49. It is understood that the shirt 1 is merely for exemplary purposes and the garment could be of other types depending on the needs of the person in a high risk activity. For instance the garment might take the form of a various garments such as a flight suit, diving or swimming wet suit, battlefield outerwear, fire fighter outerwear or police outerwear.

The shirt 1 and pants 13 preferably include a total of eight flaps 3,4,5,6,7,8,9,10 for accessing the internal tourniquet system but could include more or less flaps and tourniquets. The flaps are releasable secured to the garment by hook and loop fasters or other suitable quick release connectors. Flaps 3 and 5 are located such that the tourniquet band, See FIGS. 4 and 7-10, of the internal tourniquet system is centered at the level of superior position of the bicep. Flaps 6 and flap 4 are located such that the tourniquet band of internal tourniquet system is centered at two centimeters below the elbow of the wearer. Flaps 8 and 7 are located such that the tourniquet band of internal tourniquet system is centered at two centimeters distal to the inguinal crease and flaps 9 and 10 are located such that the tourniquet band of internal tourniquet system is centered at two centimeters distal to the tibial tuberosity.

FIG. 2 discloses an alternate embodiment of the current invention illustrated in a garment that includes a short-sleeved, button-down shirt 24 with two flaps 25, 26 for accessing the internal tourniquet system. Flaps 25 and 26 are located such that the tourniquet bands of the internal tourniquet system is centered at the level of superior position of the bicep.

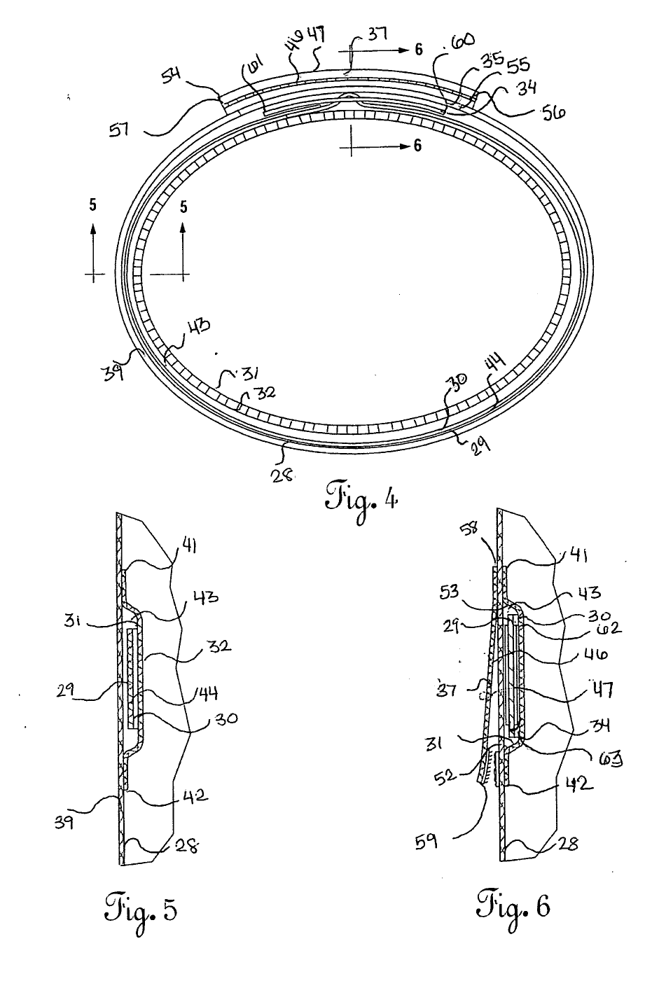

FIG. 3 discloses a suggested location and orientation for the storage location of the lever or bar 11 on the outer surface 49 of the garment during times when it isn't employed as assistance in tightening a tourniquet 23. The storage comprising of a generally rectangular webbing member 12a sown or otherwise secured to the other surface 49 of the garment and having an outer surface 12 and an inner surface 14, an upper edge 17, a lower edge 18, a right edge 19 and a left edge 20. The inner surface 14 of the webbing secured to the outer surface 49 of the garment at the lower edge 17, the right edge 19 and the left edge 20 in any suitable manner such that a channel is formed of the same size or slightly larger than the lever or bar 11 and leaving unsecured the upper edge 17 for insertion of the lever or bar 11 and storage during times of disuse. The webbing having a flap with outer surface 15 and inner surface 16, a lower edge 21 and an upper edge 22. The inner surface 16 secured to the outer surface 49 of the garment at the upper edge 22 in any suitable manner such that the lower edge 21 of the inner surface 16 of the flap overlaps the upper edge 17 of the outer surface 12 of the webbing. At the point of overlap, the lower edge 21 of the inner surface 16 of the flap and the upper edge 17 of the outer surface 12 of the webbing, having a securing means, such as convention hook and loop fasteners or other suitable quick release connection, to secure the lever or bar 11 in the storage location.

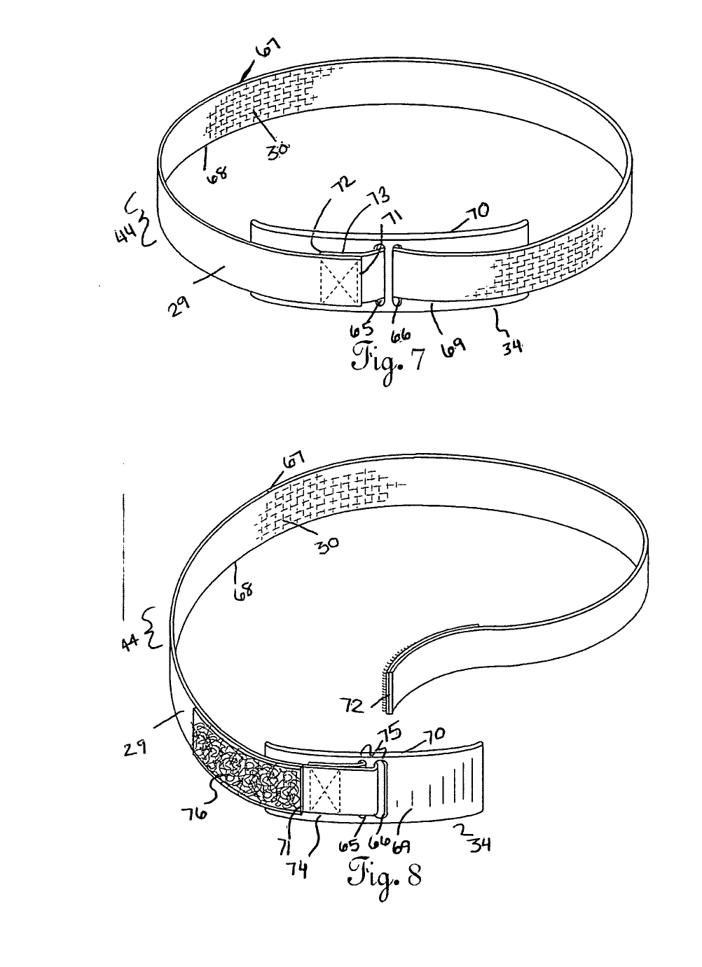

FIG. 4 discloses a horizontal cross section of an embodiment of the internal tourniquet system. The innermost continuous ring is the webbing or band 40 which has an outer surface 31 and an inner surface 32. The webbing 40 has an upper edge 41, see FIG. 6, and a lower edge 42. The fabric of the garment has an inner surface 28. The inner surface of the webbing 32 is secured at the upper edge 41 and the lower edge 42 to the inner surface of the garment 28 in any suitable manner that may include sewing or adhering or molding such that a channel 43 is formed of a slightly larger than the tourniquet band 44 and continuing the entire circumference of the inner surface of the garment 28. The tourniquet band 44, with outer surface 29 and inner surface 30 is located within the channel 43 in such a manner that the outer surface of the webbing 31 is adjacent but in no way would noticeably impede the movement of, the inner surface of the tourniquet band 30 such that when the tourniquet is tightened or loosened, it is free to move within the channel 43. The channel makes the tourniquet more comfortable and less irritation or noticeable but does not interfere with the use of the tourniquet. In the same manner, the outer surface of the tourniquet band 29 is adjacent to the inner surface of the fabric of the garment 28 wherein the inner surface of the garment 28 in no way impedes, in any noticeable manner, the movement of the outer surface of the tourniquet band 29. The tourniquet 23 has a tightening and securing mechanism 34 located such that the outer surface of the tourniquet band 29 lies beneath and loops through the tightening and securing mechanism 34 in a manner that allows for the tourniquet band circumference to be decreased in diameter thus producing a higher radial pressure on the extremity to staunch any current blood flow and allows for the decreased circumference to be maintained without constant torque from an outside source. The tightening and securing mechanism 34 lies beneath a portal 35 in the garment 39 through which the internal tourniquet system can be accessed. The portal has an upper edge 53 (See FIG. 6.), a lower edge 52 (See FIG. 6.), a left edge 55 and a right edge 54. The distance between the left edge 55 and the right edge 54 of the portal can be as large as or larger than the width of the tightening and securing mechanism 34. The width of the tightening and securing mechanism 34 is defined as the distance between the right edge of the mechanism 60 and the left edge of the mechanism 61. The portal 35 is protected by a flap 37 with inner surface 46 and outer surface 47 an upper edge 58 (See FIG. 6.), a lower edge 59 (See FIG. 6.), a right edge 56 and left edge 57. The flap inner surface 46 overlaps with the outer surface of the garment 49 below the lower edge of the portal 52 (See FIG. 6.) and the flap left edge 57 and flap right edge 56 overlap the left edge 56 and right edge 57 of the portal, respectively. At the point of overlap of the lower edge of the inner surface 46 of the flap with the outer surface of the garment 49 there is a temporary securing means 51 (such as A hook and loop fastener) to secure the flap 37 and protect the internal tourniquet system when not in use and to allow quick access when tourniquet use is required.

FIG. 5 discloses a vertical sectional view of the channel 43 taken along line 5-5 where the tourniquet band 44 is located internal to the garment 39. The webbing 40 has an upper edge 41 a lower edge 42 an inner surface 32 and an outer surface 31. The inner surface of the webbing 32 is secured at the upper edge 41 and the lower edge 42 to the inner surface of the garment 39 in any suitable manner such that a channel 43 is formed of the same size or slightly larger than the tourniquet band 44 and continuing the entire circumference of the inner surface of the garment 28. The tourniquet band 44, with outer surface 29 and inner surface 30 is located within the channel 43 in such a manner that the outer surface of the webbing 31 is adjacent but in no way would noticeably impede the movement of, the inner surface of the tourniquet band 30 such that when the tourniquet is tightened or loosened, it is free to move within the channel 43. In the same manner, the outer surface of the tourniquet band 29 is adjacent to the inner surface of the fabric of the garment 28 wherein the inner surface of the garment 28 in no way impedes, in any noticeable manner, the movement of the outer surface of the tourniquet band 29 while at the same time the webbing 40 and the fabric of the garment 39 are means of protecting the integrity of the tourniquet band 44 positioning the tourniquet band 44 within the garment.

FIG. 6 discloses a vertical sectional view of the flap 37 taken along line 6-6 which overlays and protects the tourniquet tightening and securing mechanism 34 accessible through the portal 35. The webbing 40 is connected to the garment 28 in the same manner as that described in FIG. 5. Except FIG. 6 discloses the tightening and securing mechanism 34 located such that the outer surface of the tourniquet band 44 lies beneath and loops through the tightening and securing mechanism in the manner described in FIG. 4. The distance between the upper edge 53 and lower edge 52 of the portal can be a large or larger than the height of the tightening and securing mechanism 34. The height of the tightening and securing mechanism is defined as the distance between the upper edge 62 of the tightening and securing mechanism and the lower edge of the tightening and securing mechanism 63. The portal 35 is protected by a flap 37 with inner surface 46 and outer surface 47 an upper edge 58 and lower edge 59. The inner surface of the flap 46 is secured to the outer surface of the garment 49 at the upper edge 53 in any suitable manner such that the lower edge 59 of the inner surface 46 of the flap overlaps the lower edge of the portal 52 and lies adjacent to the outer surface of the garment 49. At the point of overlap, the lower edge 59 of the inner surface 46 of the flap and the outer surface of the garment 49 have a temporary securing means, such as a hook and loop fastener to secure the flap 37 when the invention is not in use and allow easy access to the tightening and securing mechanism 34 when needed.

An important feature is that the tourniquet system of the invention can be applied with one hand. This is made possible by the mounting of the tourniquet in the garment in the correct location and making it accessible with one hand and making it possible for a person to tighten with one hand to stem blood flow. When the tourniquet is not secured on the clothing it may be difficult to attach, apply and tighten the loose tourniquet with only one hand.

FIG. 7 discloses another embodiment of a tourniquet of the current invention wherein the tightening and securing mechanism 34 attached to the tourniquet band 44 contains two openings 65 and 66. The height of the openings 65 and 66 is defined by the distance between the upper edge of the tourniquet band 67 and the lower edge of the tourniquet band 68. The openings 65 and 66 must be high enough to allow the tourniquet upper 67 and lower 68 edges to be accepted. In the same manner the width of the openings 65 and 66 is defined by the distance between the inner surface 30 and outer surface 29. The openings must be wide enough to allow the tourniquet band's inner 30 and outer 29 surfaces to pass through with no noticeable resistance. The distance between the upper 62 and lower edge 63 of the tourniquet tightening and securing mechanism 34 must only be slightly larger than the height of the openings 65 and 66. The tightening and securing mechanism 34 may be made of carbon fiber or any similar material designed to withstand the average forces and fatigue likely to be realized by the tourniquet tightening and securing mechanism 34.

The tourniquet band 44 shown in FIG. 7 may be made of nylon or any similar material designed to withstand the average forces and fatigue likely to be realized by the tourniquet. This embodiment details the tourniquet band 44 wherein the inner surface of the tourniquet band 30 lies adjacent to the outer surface of the mechanism 69 until opening 66. The band 44 is then passed through opening 66 such that the outer surface of the band 29 is adjacent to the inner surface of the mechanism 70. The band 44 is then passed through opening 65 such that the bands inner surface 30 is again adjacent to the outer surface of the mechanism 69. The left edge 71 and right edge 72 of the tourniquet band are arranged such that a significant amount of the inner surface 30 of the tourniquet at the left edge 71 overlaps the outer surface 29 of the tourniquet at the right edge 72. At the point of overlap a temporary securing means 73 (such as a hook and loop fastener) is used to close the tourniquet band 44 when not in use and to release, tighten and re-secure when in use.

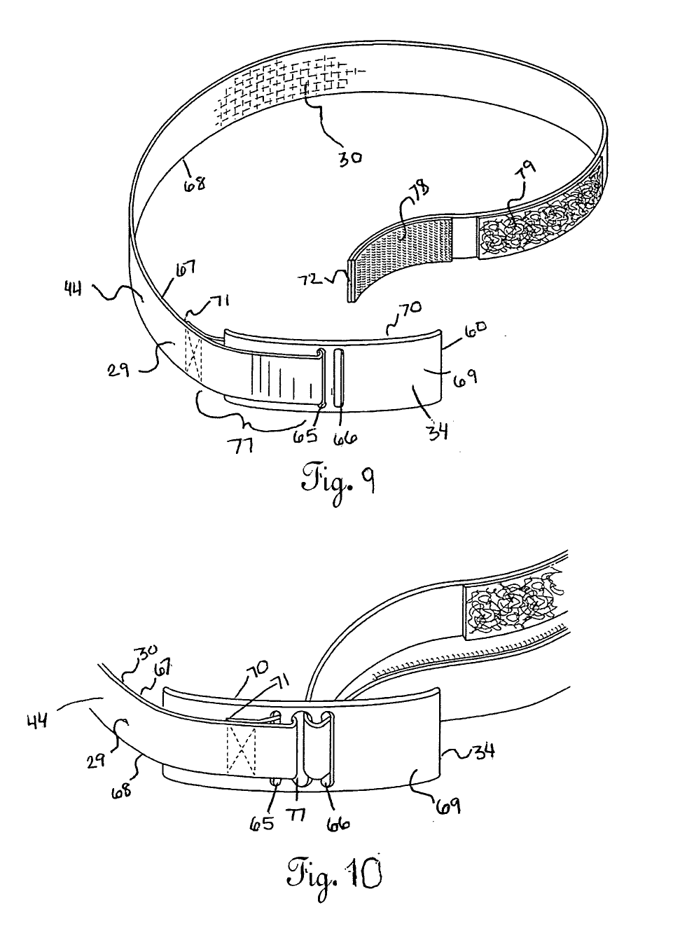

FIG. 8 discloses another embodiment of the tourniquet of the current invention. The tightening and securing mechanism 34 is formed in the same manner however the tourniquet band 44 is arranged in a different configuration. In this embodiment the left edge of the tourniquet band 71 is passed through opening 66 such that the inner surface of the band 30 is adjacent to the inner surface of the mechanism 70. The left edge 71 is then passed through opening 65 such that the inner surface 30 of the left edge 71 of the band is now adjacent to a portion of the inner surface of the band 30 farther from the left edge 74. At this point the tourniquet band is secured to itself in any suitable manner such that a loop 75 is formed near the left edge 71 of the tourniquet wherein the tightening and securing mechanism 34 is contained within the loop 75. The outer surface of the tourniquet band 29 adjacent to the point where the tourniquet is secured to itself 74 contains one end of a temporary securing means 76 (such as a hook and loop fastener). Along the inner surface 30 of the tourniquet band near the right edge 72 lies the matching end of the temporary securing means such that the right edge is passed through the tightening and securing mechanism opening 66 in a manner that before passing through the band's right edge 72 outer surface 29 is adjacent to the inner surface of the mechanism 70 and after passing through the inner surface 30 of the right edge 72 overlaps the temporary securing means 76 such that the temporary securing means may be used to tighten, secure, release and re-secure the tourniquet as needed.

FIG. 9 discloses another embodiment of a tourniquet for the current invention wherein on tightening and securing mechanism 34 is formed in the same manner as FIGS. 7 and 8. However, the tourniquet band 44 is affixed to the mechanism 34 in a differing manner. In this embodiment the inner surface 30 of the left edge 71 is passed over the outer surface 69 of the mechanism. The left edge 71 is then passed through opening 65 in a manner such that the inner surface 30 of the left edge 71 then becomes adjacent to the inner surface 70 of the mechanism until it meets and overlaps another portion of the inner surface 30 of the band. At the point of overlap the inner surfaces are secured to each other in any suitable manner such that a loop 77 is formed containing wholly half of the tightening and securing mechanism 34.

Near the outer surface 29 of the right edge 72 of the tourniquet band lie two opposing ends of a temporary securing system (such as A hook and loop fastener) 78, 79. The tourniquet is activated by passing the right edge 72 of the band through the opening 66 such that after passing through opening 66 the outer surface 69 of the right edge 72 adjacent to the outer surface 69 of the tightening and securing mechanism. The outer surface 29 of the right edge 72 should pass beyond the right edge of the mechanism 60 and overlap the outer surface of the band 29 where the opposing temporary securing means 79 is located in order to secure the tourniquet when needed.

FIG. 10 discloses another embodiment of the tightening and securing mechanism of the current invention. The mechanism is formed in the same manner as noted in FIG. 7-9. However, an additional opening 77 in between openings 65 and 66 with the same height as those in FIGS. 7-9 is present. Opening 77 must have a width at least as great as twice the distance between the inner surface 30 and outer surface 29 of the tourniquet band and must allow two tourniquet bands 44 to pass through at the same time with no noticeable resistance. Loop 75 as in FIG. 7 is again created using openings 65 and 77. However, the right edge 72 (See FIG. 8.) of the tourniquet band is passed through opening 77 such that the outer surface 29 of the right edge 72 is adjacent to the outer surface 69 of the tightening and securing mechanism. The right edge 72 is then passed through opening 66 such that the outer surface 29 of the tourniquet band is now adjacent to itself and the inner surface of the tourniquet band 30 is adjacent to the inner surface 70 of the tightening and securing mechanism. Placed upon the overlap of the outer surface 29 of the right edge 72 of the tourniquet band before and after the band has been threaded through the openings 77 and 66 is a temporary securing means 78, 79 such that the overlap may be tightened, secured and released when needed.

While the releasable bands shown in FIGS. 7-10 are preferred, a convention band that required twisting by the use of bar 11 or other suitable lever could be used. The medical use of the tourniquet system to treat a patient would be the generally the same as with any conventional tourniquet but the invention would make the tourniquets immediately available for use to stop life threatening blood loss.

The use of the tourniquet system of the invention would be as follows. If an appendage of a wearer of the garment was injured and a tourniquet force was needed to stop bleeding the medic or person treating the injured wearer would simply lift the flap 7 to expose the tourniquet band 30. The medic would then apply tourniquet pressure to the bank 30 to stop the blood loss. The type of pressure applied would be the same as with other standard tourniquets based on the need of the patient. If needed the flap 15 could be released to expose the bar 11. The bar 11 could be removed from the pouch 12 and inserted in the tourniquet and twisted to apply additional pressure as in conventionally done with tourniquets.

In all FIGS. 7-10 the lever or bar 11 may be inserted between the inner surface 29 of the tourniquet band and the outer surface 31 of the mesh at the portal 35 and twisted either clockwise or counterclockwise in an effort to tighten the tourniquet band 44 to an even further degree. Once tightened the lever or bar 11 may then be secured in place by securing flap 37.

Although a system and method according to the present invention have been described in connection with the preferred embodiments, the present invention is not intended to be limited to the specific forms set forth herein, but, on the contrary, it is intended to embrace such alternatives, modifications, and equivalents, as can be reasonably included within the spirit and scope of the foregoing description. The scope of the invention is limited only by the appended claims.

We claim:

1. An internal tourniquet system for use by a person in a high risk activity comprising:
a circumferential enclosed channel formed in an appendage portion of the garment;
a tourniquet with a tightening and securing mechanism positioned in the circumferential enclosed channel on the appendage portion of the garment for applying a tourniquet force to the inside wall of the circumferential enclosed channel; and
a portal in the garment placed on the circumferential enclosed channel to provide access to the tourniquet for allowing operating the tightening and securing mechanism of the tourniquet.

2. An internal tourniquet system as recited in claim 1, further comprising:
a removable flap with temporary securing means placed over the portal in the garment for covering the portal during non-use of the tourniquet.

3. An internal tourniquet system of claim 1, further comprising:
the circumferential enclosed channel being located on the centerline of the first tourniquet on the upper extremities at the superior position of the bicep.

4. An internal tourniquet system of claim 1, further comprising:
the circumferential enclosed channel being located on the centerline of the second tourniquet on the upper extremities at two centimeters below the elbow of the user.

5. An internal tourniquet system of claim 1, further comprising:
the circumferential enclosed channel being located on the centerline of the first tourniquet on the lower extremities at two centimeters below the inguinal crease.

6. An internal tourniquet system of claim 1, further comprising:
the circumferential enclosed channel being located on the centerline of the second tourniquet on the lower extremities at two centimeters distal to the tibial tuberosity.

7. An internal tourniquet system of claim 2, further comprising:
a bar to apply additional torque to the tourniquet band; and
a storage location on the garment for the bar when not in use.

8. An internal tourniquet system of claim 2, further comprising:
a removable flap covering to secure the bar in position once desired torque is reached.

9. A method for protecting a person in a high risk activity using an internal tourniquet system comprising the steps of:
forming a circumferential enclosed channel in an appendage portion of the garment;
positioning a tourniquet with a tightening and securing mechanism in the circumferential enclosed channel on the appendage portion of the garment for applying a tourniquet force to the inside wall of the circumferential enclosed channel; and
forming a portal in the garment on the circumferential enclosed channel to provide access to the tourniquet for allowing operating the tightening and securing mechanism of the tourniquet.

10. The method as recited in claim 1, further comprising:
forming a removable flap with temporary securing means over the portal in the garment for covering the portal during non-use of the tourniquet.

11. The method as recited in claim 1, further comprising:
positioning the circumferential enclosed channel on the centerline of the first tourniquet on the upper extremities at the superior position of the bicep.

12. The method as recited in claim 1, further comprising:
positioning the circumferential enclosed channel on the centerline of the second tourniquet on the upper extremities at two centimeters below the elbow of the user.

13. The method as recited in claim 1, further comprising:
positioning the circumferential enclosed channel on the centerline of the first tourniquet on the lower extremities at two centimeters below the inguinal crease.

14. The method as recited in claim 1, further comprising:
Positioning the circumferential enclosed channel on the centerline of the second tourniquet on the lower extremities at two centimeters distal to the tibial tuberosity.

15. The method as recited in claim 1, further comprising:
forming a storage location on the garment for a bar for the tourniquet band when not in use.

16. The method as recited in claim 1, further comprising:
forming a removable flap covering to secure the bar in position once desired torque is reached.
